(19) United States
(12) Patent Application Publication
(10) Pub. No.: US 2014/0275795 A1
(43) Pub. Date: Sep. 18, 2014

(54) ACCESS DEVICE WITH VARIABLE LUMEN

(71) Applicants: Eric Forrest Little, Shakopee, MN (US); Richard Duda, Stillwater, MN (US); Wade R. Eichhorn, Minneapolis, MN (US); Kristian G. Wyrobek, Minneapolis, MN (US)

(72) Inventors: Eric Forrest Little, Shakopee, MN (US); Richard Duda, Stillwater, MN (US); Wade R. Eichhorn, Minneapolis, MN (US); Kristian G. Wyrobek, Minneapolis, MN (US)

(73) Assignee: 7-SIGMA, INC., Minneapolis, MN (US)

(21) Appl. No.: 14/208,575

(22) Filed: Mar. 13, 2014

Related U.S. Application Data
(60) Provisional application No. 61/784,540, filed on Mar. 14, 2013.

(51) Int. Cl. A61B 17/34

(52) U.S. Cl. CPC A61B 17/3423
USPC 600/208

(57) ABSTRACT

A device allows access into a body has at least:
A first cannula with an outer surface, an inner lumen surface, an inner lumen diameter and proximal and distal end portions; and
An expandable covering over the outer surface, the expandable covering having an outer surface and an inner surface; and
The inner surface of the expandable covering remaining in contact with the outer surface of the first cannula.

### CROSS REFERENCE TO RELATED APPLICATIONS

This application claims priority under U.S. Law from U.S. Provisional Patent Application Ser. No. 61/784,540, filed Mar. 14, 2013, 2013.

### FEDERALLY SPONSORED RESEARCH

None

### SEQUENCE LISTING

None

### BACKGROUND OF THE INVENTION

1. Field of the Invention

The present disclosure relates to methods and devices for creating access and communication paths between at least two regions separated by a barrier and more particularly to the introduction of diagnostic or therapeutic catheters into vasculature and other body compartments.

2. Background of the Art

The present disclosure pertains to the field of sheaths for introducing catheters into a body. In particular, the present disclosure relates to a flexible sheath for percutaneously introducing intravascular catheters such as an angioplasty catheter.

The Seldinger technique is a method commonly used to gain percutaneous access to a patient's vasculature and is described in detail by U.S. Pat. Nos. 7,699,864; 4,362,150 and 6,183,443 “Expandable Introducer Sheath” both incorporated in entirety by reference herein. The Seldinger technique involves inserting a needle through a patient's skin and into the lumen of a blood vessel, such as an artery or vein. A flexible guide wire is then advanced through the needle into the blood vessel to define a pathway through the skin into the vessel lumen leaving a proximal end of the guide wire protruding from the body. Next, a practitioner prepares an introducer sheath by inserting a distal end of a dilator (e.g., an elongate flexible cylinder with a bore extending therethrough) into a proximal end of a flexible introducer sheath and advances the dilator fully into the introducer sheath until a distal tip portion of the dilator extends distally beyond a distal end of the sheath and a proximal portion of the dilator remains outside the proximal end of the sheath. The distal tip portion of the dilator has a tapered outer diameter that gradually increases proximally to a diameter that is approximately equal to the distal end of the sheath. The proximal portion of the dilator is releasably secured to the proximal portion of the sheath so that the dilator and sheath comprise an assembled unit for insertion into the vessel.

The distal end of the dilator (with the sheath loaded thereon) is then threaded over the proximal end of the guide wire and inserted through the skin and underlying tissue into the vessel lumen by distally advancing the dilator and sheath over the wire. As the dilator is longer than the sheath, the distal end of the dilator enters the vessel before the distal end of the sheath and the dilator and sheath are advanced together until the distal portion of the sheath extends into the vessel. The tapered distal tip portion of the dilator gradually expands the opening in the vessel wall as the dilator moves there through. With the distal end of the sheath properly positioned and extending into the vessel, the proximal end of the sheath and the proximal end of the dilator remain outside the surface of the skin. Next, while maintaining the sheath in place within the vessel and after disengaging the dilator from the sheath, the dilator and wire are removed by proximally withdrawing them from inside the sheath. With the sheath in place, a practitioner is afforded convenient and protected access to the patient's vasculature for the introduction of diagnostic and/or therapeutic devices.

The sheath facilitates the insertion and withdrawal of intravascular devices through the skin and underlying tissue into a vessel while minimizing trauma to the skin puncture site and vessel wall caused by the insertion and removal of intravascular devices from the vessel. Further, the introducer sheath prevents back-bleeding, or blood flow exiting the punctured vessel, since the sheath generally has a hub attached therein at its proximal end. The hub can be any form of fluid control device which includes, but is not limited to, a hemostasis valve. The hemostasis valve forms a fluid-tight seal around the diameter of intravascular devices including catheters and guide wires to prevent a flow of blood out of the patient or air into the patient. The hemostasis valve also provides proximal end sealing of the sheath when no device extends through the hemostasis valve.

Although the inner diameter of the sheath should have a close tolerance with the outer diameter of the intravascular device, it is desirable to have some spacing between the sheath and intravascular device to minimize friction and ‘windup’, where windup is the resultant angular rotation between the proximal and distal ends of an intravascular device in response to an applied torque, to allow for improved motion control of the intravascular device. Further, the spacing can be desirable for perfusion or for drug infusion flow techniques through that spacing. The hub can thus also take the form of a side arm with a 3-way valve connector to allow for blood perfusion or drug infusion.

Reasons for minimizing the size of a sheath include minimizing the size of the opening in the vessel and the skin puncture site, increasing the stability of the sheath within the skin puncture site, and reducing the time for this puncture site to heal. There are two reasons that this time is of interest. First, ensuring the proper clotting of this opening requires the attention of trained personnel for several minutes (e.g., 5 to 15 minutes) after the sheath is removed. Second, patients need to remain immobile for many hours after the sheath is removed to ensure that the clotted opening in the vessel does not reopen. These healing times can be long because patients may have an anticoagulant in their cardiovascular systems. Hence, it is desirable to reduce both of these times.

Although it is desirable to minimize the outer diameter of the introducer sheath, an intravascular device having an outer diameter larger than the inner diameter of the introducer sheath already in place may be required later in the surgical procedure. These larger size intravascular devices require the use of a larger size introducer sheath and accordingly, necessitate exchanging the first introducer sheath for another introducer sheath having a larger inner diameter.

For example, this situation frequently arises because a smaller size introducer sheath is required for angiography procedures and a larger size introducer sheath is required for an angioplasty procedure. For example, a procedure using angiography catheters typically would be performed with an introducer sheath having a size 5 to 6 French (1.67 to 2 mm) inner diameter. However, present day angioplasty guide catheters through which an angioplasty dilation catheter would pass are generally too large to fit through size 5 to 6 French (1.67 to 2 mm) introducer sheaths. Accordingly, if it were determined that an angioplasty procedure was required, then a larger inner diameter size introducer sheath, for example 7 to 8 French (2.33 to 2.67 mm) would be needed to accommodate the outer diameter of an angioplasty guide catheter. If an adjunctive procedure such as an atherectomy, thrombectomy or stent placement procedure would be necessary after or instead of the angioplasty procedure, an even larger size introducer sheath would be required.

With the possibility of these different sized introducer sheaths being required, the physician is faced with a dilemma. It is highly desirable to use the smallest size introducer sheath possible to minimize the size of the opening in the skin and in the vessel (e.g., femoral artery). However, if one selects an introducer sheath that is too small to accommodate all the necessary intravascular devices, then the smaller size introducer sheath would have to be later exchanged for a larger one. Confronted by this choice, physicians may choose to insert an introducer sheath of a size large enough to easily accommodate all potential intravascular devices. This means that an introducer sheath may be selected that is much larger than necessary and this initial choice for the larger introducer sheath may sacrifice the highly desirable goal of minimizing the size of the opening in the artery wall and skin puncture site.

In a case where a smaller inner diameter size sheath was initially selected and must be removed to be replaced by a larger inner diameter size sheath, all intravascular devices from within the vessel typically must be removed with the exception of a guide wire. Next, the smaller size sheath must be removed from the vessel and skin surface puncture site. To do so, with the sheath still in place within the vessel, the physician reinserts the dilator into the sheath until the dilator extends within the vessel beyond the sheath (and the sheath locks with the dilator) so that the wire may be threaded through the dilator until the distal end of the wire extends through the vessel distally beyond the distal end of the dilator. While leaving the wire in place within the vessel, the dilator and sheath are removed from within the vessel.

Next, to place a larger introducer sheath within the vessel, a physician would repeat the entire percutaneous puncture insertion method for introducer sheaths as previously described (except for not using a puncture needle because the wire already extends the vessel). If this procedure is performed at a new puncture site along the vessel, then a new puncture site would be needed. In any case, repeating the percutaneous insertion procedure traumatizes the endothelium layer of the vessel wall, the surrounding tissue, and the skin much more than performing the percutaneous insertion technique only once. Moreover, many patients receiving angioplasty treatment already suffer from diseased arterial walls which magnify the problem of repetitious trauma to vessel wall. Such trauma further increases the risk of hematoma or dislodging of plaque creating emboli which can lead to catastrophic events such as cerebrovascular accident (e.g., stroke).

Because of the large number of devices of varying sizes which may be used in a combined angiography/angioplasty, or adjunctive procedure, the conventional introducer sheath has many deficiencies. One major deficiency is that once having been inserted, there is no mechanism for increasing the size of the introducer sheath other than by replacing the smaller size introducer sheath with a second larger size introducer sheath through a second percutaneous insertion procedure. This deficiency may drive the physician to reluctantly select an introducer sheath with a size potentially much larger than necessary, needlessly increasing the size and healing time of the opening created in the vessel wall and skin surface. This can result in longer patient recuperation time which can require an overnight stay in the hospital for a procedure that otherwise could be done on an outpatient basis.

Various attempts have been made to improve the shortcomings of traditional non-expanding introducer devices. For example, U.S. Pat. No. 4,921,479 (Grayzel) incorporated by reference herein, is directed to a removable, expandable sheath for introducing catheters. The sheath is made of a semi-stiff plastic with memory and formed in a tubular configuration with a longitudinal slit extending along the entire length of the sheath. The tubular structure is typically coiled about its longitudinal axis so its tubular wall overlaps itself. Upon insertion of a larger diameter intravascular device, the tubular sheath enlarges its inner diameter by uncoiling to the extent necessary to accommodate the catheter inserted therein. The Grayzel device is disadvantageous because the slit extending the length of the sheath permits potential back-bleeding. Further, the moveable nature of the walls relative to each other can traumatize the vessel possibly causing a dissection of the vessel wall or at least exacerbating theinjury to the endothelial layer of the vessel wall and the skin tissue.

U.S. Pat. No. 4,411,655 (Schreck) incorporated by reference herein, describes a needle-cannula device with an expandable radius to accommodate large bore medical diagnostic and therapeutic instruments. Lumen expansion is realized solely through the use of shape memory alloys (or SMA) which expands and contract with changes in temperature. However, the use of body temperature to dilate the '655 device restricts expansion capability and limits the bore diameter of catheter instruments that can be used. Further, since body temperature may vary during surgery, the Schreck device does not provide a reliable means for lumen diameter control. Alternatively, Schreck teaches the use of external energy sources such as resistance heating and radiofrequency induction to generate device expansion, however, such energy sources require additional equipment and can add considerable complexity and expense to the surgical procedure.

U.S. Pat. No. 7,699,864 describes an access device comprises a thin-walled sheath that is insertable into a patient through a small surgically created incision. The incision may be created using a cutdown or a percutaneous method such as that known as the Seldinger technique. Once inserted and advanced to the target surgical site, the sheath is selectively, and controllably, expanded to a desired diameter. The thin wall of the sheath is fabricated from a rectangular piece of material such as metal or plastic with two cut edges. The rectangular piece of metal or plastic is rolled tightly to create the small diameter configuration that is inserted into the patient. A cam or control member is affixed to the innermost edge of the rectangular piece of metal or plastic. The control member extends to the proximal most portion of the sheath. By rotating the control member, the operator causes the thin wall piece of rolled material to unfurl into a larger or smaller diameter, depending on the direction of rotation. A mechanical lock at the distal end of the sheath permits the control member to be selectively constrained from rotation and thus lock the sheath diameter in place.

U.S. Pat. No. 7,927,309 (Palm) herein incorporated by reference, describes an expandable catheter sheath that does not foreshorten on radial expansion. Lumen dilation is realized using an expanding layer with a stent-like mesh structure that resists radial collapse. However, as the Palm device does not collapse, on withdrawal of the device from the body, tissue at the insertion site is exposed to a relatively large sheath cross-sectional surface area such that increased resistance may result in greater trauma to the vessel and surrounding tissue.

There exists a need for an introducer sheath device that can expand and collapse in a controllable manner.

### SUMMARY OF THE INVENTION

A sheath introducer device or cannula introducing has at least a first tube with an expandable lumen and optionally a hub and a second tube of sufficient rigidity to define a clear path into the vasculature and optionally a hub. The first tube acts as a flexible conduit into the vasculature and may be expanded from a first diameter to a second diameter by inserting the second tube into the lumen of the first tube wherein the first tube can be made to expand and contract in a controllable manner.

### BRIEF DESCRIPTION OF THE DRAWINGS

FIG. 1 is a cross-section of an embodiment of the cannula tube (top) and EGD (bottom).

FIG. 2 is a cross-section of an embodiment of the cannula tube inserted into the EGD.

FIG. 3 is a cross-section of an embodiment of the cannula tube (top) and EGD with a flared proximal end portion (middle) and the complete assembly with the cannula tube inserted into the EGD (bottom).

FIG. 4 is an embodiment of the single wall thickness cross-section with live hinges.

FIG. 5 is an embodiment of the single wall thickness cross-section with alternating relatively stiff and relatively elastic segments.

FIG. 6 is an embodiment of a multi-layer cross-section the backing layer has overlapping outer surfaces covered by an expandable membrane.

FIG. 7 is an embodiment where at least two shell segments are encased in an elastic layer.

FIG. 8 is an embodiment where multiple shell segments are encased in an elastic layer.

FIG. 9 is an embodiment where at least two shells nest within each other, where the outer surfaces of one contact the inner surfaces of another.

FIG. 11 is an embodiment similar to FIG. 9 but with three shell segments using a tongue-in-groove arrangement with an elastic layer.

FIG. 12 is an embodiment of a repeating surface texturing (two instances of the repeated pattern shown) that can be applied to all surfaces of the device to minimize the growth of infection causing organisms

FIG. 13 is an embodiment of a disinfecting waveguide introducer sheath.

FIG. 14 is an embodiment of the steps used to form FIG. 13.

FIG. 15 is an embodiment of a sheath with tapered tip (left) and an isometric of an embodiment of the tapered tip.

FIG. 16 is an embodiment of a self-stenting introducer sheath.

FIG. 17 is an embodiment of an EGD with a relatively rigid lubricious coating.

FIG. 18 is embodiments of an cannula tube removable from a catheter during a procedure.

FIG. 19 is an embodiment of an isometric view (left) and cross-sectional view (right) of the spring-encased EGD embodiment.

FIG. 20 is an embodiment of a coiled backing layer.

FIG. 21 shows an embodiment of the EGD with a distal end portion that presents a square leading edge.

FIG. 22 shows an embodiment of the EGD with a distal end portion that presents a tapered leading edge.

FIG. 23 shows an example of an expanding sheath comprising an EGD and a tapered tip.

FIG. 24 shows an embodiment of a tip shell with a tapered distal end, tapered distal lumen and an untapered proximal end.

FIG. 25 shows an embodiment of a stenting device with a generally circular cross-section that has a removed portion to allow the passage of a catheter into the lumen of a stenting device.

FIG. 26 shows an embodiment of a stenting device with a generally circular cross section and a removable segment with the addition of a removable segment.

FIG. 27 shows a shell-in-shell design as an example of an external feature.

FIG. 28 shows an embodiment of the stenting device with a generally circular cross section and at least one weakened or breakaway seam.

FIG. 29 shows an embodiment of the stenting device with a generally circular cross section and at least one gap and a live hinge.

FIG. 30 shows an embodiment of the stenting device with a gap cover.

FIG. 31 shows a thin sheet of material into which a series of V-shaped perforations have been created.

FIG. 32 shows the structure of one of the V-shaped perforations comprising a tooth and a corresponding hole.

FIG. 33 shows a section view of a V-shaped perforation where the tooth has been formed in a curved fashion and set such that a tooth bent into the plane of the thin material will naturally return to its curved position after release of the bending force.

FIG. 34 shows an embodiment where a repeating surface texturing is applied to a surface of the device to minimize the growth of infection causing organisms.

FIG. 35 shows an embodiment of an EGD design used as a waveguide for radiation (such as UV light) to kill infection causing organisms.

FIG. 36A shows a manufacturing process step of 1) obtaining a tube-like piece of material appropriate to transmit radiation along its length.

FIG. 36B shows a manufacturing process step of 2) flattening a portion of the tube-like piece and trimming appropriately.

FIG. 36C shows a manufacturing process step of 3) rolling the flattened portion into a tube that may overlap so that the waveguide head and rolled tube are as shown in the side and end views.

FIG. 36D shows typical side (left) and end (right) views of the radiation waveguide tube of FIG. 35.

### DETAILED DESCRIPTION OF ILLUSTRATIVE EMBODIMENTS

The following description should be read with reference to the drawings wherein like reference numerals indicated like elements throughout the several views. The detailed description and drawings illustrate example non-limiting specific embodiments of the generic claimed invention.

The embodiments of this disclosure, while primarily directed to interventional vasculature access applications, will also find use in other medical fields including, but not limited to, urology and emergency room (or ER) medicine.

### Basic Structure

FIG. 1 shows an example of the components of the expanding sheath introducer 100 in an unexpanded state. The example includes an expanding guidepath device (or EGD) 101 comprising an expanding barrel 103 with a lumen 105 of a first diameter D1, a proximal end portion 107 and a distal end portion 109. In some embodiments, an expanding barrel hub 111 can be attached to the proximal end portion 107, the distal end portion 109 or both end portions. The example also includes a stenting device 151 with an outer diameter E (where the first diameter D1 of lumen 105 is smaller than the outer diameter E) comprising a stenting barrel 153, a proximal end portion 155 and a distal end portion 157. In some embodiments, a stenting barrel hub 159 can be attached to the proximal end portion 155, the distal end portion 157 or both end portions.

To expand the EGD 101 from an initial diameter D1 to a second diameter D2 (where D2>D1), the stenting device 151 of a desired size is selected and inserted into the proximal end portion 107 of the EGD 101. The stenting device 151 is advanced into the EGD 101 until a distal end portion 157 of the stenting device 151 extends distally beyond a distal end portion 109 of the EGD 101 and a proximal end portion 155 of the stenting device 151 remains outside the proximal end portion 107 of the EGD 101. The distal end portion 157 can have a square or tapered tip. The proximal end portion 155 of the stenting device 151 is releasably secured to the proximal end portion 107 of the EGD 101 so that the stenting device 151 and EGD 101 comprise an assembled expanding sheath introducer 100 for insertion into the vessel.

The sequential withdrawal and replacement of progressively larger diameter stenting devices 151 allows the medical practitioner to easily upsize the sheath lumen 105 to accommodate larger bore medical devices without having to replace the sheath. The ability of the expanding sheath device 100 to increase lumen diameter with the simple replacement of the stenting device 151 greatly minimizes the patient trauma associated with the insertion and removal of a series of progressively larger diameter standard sheath introducers to achieve the same result. The unique design of the EGD 101 also allows medical practitioners to downsize the sheath lumen 105 as will become apparent from the disclosure.

FIG. 2 shows an example of the EGD 101 in an expanded state where the stenting device 151 can be inserted into the lumen 105 of the unexpanded EGD 101 whereafter the lumen 105 expands to a second diameter D2 which is larger than first diameter D1 and generally conforming to the shape of stenting barrel 153.

Referring again to FIG. 1, in some embodiments, the expanding barrel hub 111 and stenting barrel hub 159 can be a hemostatic valve. Hemostasis valves are well-known in the industry and examples are disclosed in U.S. Pat. No. 4,798,594 and U.S. Pat. No. 4,895,565 which are incorporated herein by reference. In another embodiment, the expanding barrel hub 111 and stenting barrel hub 159 can include a side port with attached tubing so that fluid may be introduced into the sheath introducer.

FIG. 3 shows an embodiment of the expanding sheath introducer 100 in an expanded state where the proximal end portion 107 of the EGD 101 may include a flared portion 312 of a diameter greater than the diameter of the stenting device 151. The expanding barrel hub 111 can be attached to the flared portion 312. This embodiment has the advantage of allowing designers to size the expanding barrel hub 111 to limit the diameter of the stenting device 151 that is inserted into the lumen 105 of the EGD 101. The flared portion 312 can be formed by any suitable approach as will be recognized by those skilled in the art.

### Single Layer EGD Embodiments

The EGD 101 can be formed from materials whose material properties are generally uniform across the wall thickness of a given cross-section perpendicular to the long axis of the EGD 101. FIG. 4 shows an embodiment of the EGD 101 cross-section with more than one live hinge 410. As the stenting device 151 is inserted into the EGD 101, radial forces generated by the impingement of the outer diameter of 151 on the inner diameter of 101 will tend to flatten out the live hinge 410 thereby allowing the EGD 101 to expand to the outer diameter of the stenting device 151.

FIG. 5 shows an embodiment where the EGD 101 cross-section comprises at least one relatively elastic wall segment 511 and at least one relatively inelastic wall segment 513. The EGD 101 shown in FIG. 5 can be manufactured using several techniques including extrusion over-molding. As the stenting device 151 is inserted into the EGD 101, radial forces will tend to stretch the relatively elastic wall segments 510 thereby distributing the expanding forces over a larger area so as to allow EGD 101 to expand and conform to the outer diameter of the stenting device 152.

FIG. 6 shows an embodiment of the EGD 101 comprising a coiled wire 610 encased in an elastic sleeve 620. Depending on the pitch of the coils, the coiled wire 610 acts as backing to prevent wall puncture of the EGD 101 on insertion of the stenting device 151 into lumen 105. In one embodiment the coiled wire 610 is treated with a lubricant prior to applying the elastic sleeve 620 thereby allowing the coiled wire 610 to assume a larger radius when subjected to radial forces while minimizing friction due to the motion of the coiled wire 610 against the elastic sleeve 620 generated as a result of insertion of the stenting device 152 into lumen 105 of EGD 101. The EGD 101 of FIG. 6 can be manufactured by a variety of processes including, but limited to, forming the coiled wire 610 from wire stock wrapped around a mandrel and then dip-molded or extrusion processed to encapsulate the coiled wire 610 in the elastic sleeve 620.

### Multiple Layer EGD Embodiments

The EGD 101 can be formed from at least two layers of materials typically comprising a backing layer generally capable of resisting wall puncture by instruments introduced into the lumen 105 and an elastic layer capable of expanding from an initial diameter D1 to a second diameter D2 (where D2>D1) under radial forces applied to lumen 105 and then returning to the approximately initial diameter D1 after removal of the radial forces. The radial forces required to expand the elastic layer can be imparted to the lumen 105 by inserting the stenting device 151 into the lumen 105.

Development of the multi-layer EGD 101 embodiments is rooted in the realization that an introducer sheath requires a finite amount of columnar rigidity (e.g., resistance to column buckling) for insertion of the sheath into a vesicle, however, once in the vesicle, the sheath need only provide a through hole (or radial rigidity) to allow for the easy insertion, removal and manipulation of catheter-like medical devices. As such, a device that features a structure that allows for radial expansion and contraction yet can simultaneously provide columnar and radial rigidity would be of great benefit to patients undergoing percutaneous surgical procedures.

Radial forces are necessary to expand the EGD 101 to a second diameter D2 that is larger than the initial diameter D1 (i.e., D2>D1). Minimization of the radial forces required to expand the EGD 101 is desirable as the linear forces necessary for insertion of the stenting device 151 into the EGD 101 will also be minimized resulting in less effort by the medical practitioner to insert the stenting device 151. Further, the wall thickness of the EGD 101 and the stenting device 151 should be minimized to avoid trauma to the vesicle, yet be stiff enough to resist ‘kinking’ or failure of the tube to maintain a generally circular lumen cross section when applying a bending moment to the device. An important parameter in the design of such a device is the polar moment of inertia, or that moment of inertia about the central diameter of the generally cylindrical tube that forms the EGD 101.

Moments of inertia can be thought of broadly as a structure's resistance to bending. This can be understood by way of experience; it is much easier to bend a paperback book than an equally-sized hardcover book because the moment of inertia of the hardcover book (with its rigid covers at a distance from the neutral bending axis) is much greater than that of a paperback book. A similar phenomenon is at work with regards to a tube; for a given wall thickness, a ‘large’ diameter tube will have a greater bending resistance than a ‘small’ diameter tube. From a design standpoint, it is desirable for cross sectional areas to have larger moments of inertia than smaller to avoid kinking failures.

A structure's ability to resist bending is also dependent upon the shear forces that can be distributed about the cross-section of a given beam. Thus, an open section circular tube will have less bending resistance than a closed section circular tube of the same diameter. Note that the closed section tube can distribute bending loads evenly about the entire cross-sectional area whereas the open tube will experience stress concentrations, or regions of focused bending forces, at the ends of the open sections. This will lead to local material yield and structural failure in the open section tube before that of a closed section tube all other parameters being equal. From a design standpoint, it is therefore desirable to build tubes with closed cross-sections as opposed to open cross-section to avoid kinking failures.

Experimental measurement of standard sheath introducer assemblies reveals that many of these devices have a sheath wall thickness of from 1-2 Fr (0.33-0.67 mm). Given their wide use in the medical industry, it can be assumed that the aforementioned sheath wall thickness is adequate and devices constructed with this approximate thickness in mind will be readily accepted by medical practitioners. However, to operate as an expanding sheath, a multi-layer sheath design must utilize an open cross-section to allow for growth of the sheath diameter while minimizing the required radial forces to expand the device which decreasing bending strength.

An additional realization was that the drawbacks of an open cross-section tube can be mitigated with the use of multiple open cross-section tubes working together. As noted previously, shear stresses experienced during bending moments are more uniformly distributed in closed section cross-sectional areas than in open section cross-sectional areas. However, various combinations of at least two open section cross-sectional area shells as shown, for example, in FIGS. 7-18, can overcome the lack of a single structure to resist shear stress by instead relying on frictional forces between at least two shells when held closely together with the elastomeric layer to approximate the bending resistance of a closed section cross-section. Experiments on various prototypes have demonstrated that the presence of frictional interfaces between at least two shells is, in fact, predictive of increased bending stress.

FIG. 7 shows a cross-sectional view of an embodiment of the EGD 101 where the backing layer is a shell 710 formed from one continuous sheet of thin material with an overlapping segment 712 and a continuous elastic layer 720. The EGD 101 of FIG. 7 can be manufactured by a variety of processes including, but not limited to, forming the shell 710 on a mandrel of generally circular cross-section from a sheet of material which is heated to allow the shell 710 to take the shape of the mandrel and left to cool afterwhich the shell 710 is dip-molded or extrusion processed to encapsulate the backing layer 710 in the elastic layer 720. In some embodiments, at least a portion of the backing layer 710 is treated with a release agent so that the elastic layer 720 will not adhere to the backing layer 710. In this manner, radial forces required to cause the EGD 101 to expand from a first diameter to a second diameter will be minimized as elastic layer 720 displacement will be more uniformly distributed around the periphery of the backing layer 710.

FIG. 8 shows an embodiment similar to FIG. 7 where the backing layer can be a shell 810 formed from one continuous sheet of thin material in a generally circular shape with an overlapping segment 812 and an elastic layer 820 disposed between the two edges of the overlapping segment 812. In other embodiments, the elastic layer 820 adhesion area can be controlled or allowed to be something less than or greater than the entire overlapping area.

FIG. 9 shows an example where the backing layer can be a shell 910 formed from one continuous sheet of thin material in a generally circular shape where the edges of the shell 910 do not overlap and an elastic layer 920 disposed continuously around the shell 910. The EGD 101 of FIG. 9 can be manufactured by a variety of processes including, but not limited to, forming the shell 910 on a mandrel of generally circular cross-section from a sheet of material which is heated to allow the shell 910 to take the shape of the mandrel and left to cool afterwhich the shell 910 is dip-molded or extrusion processed to encapsulate the backing layer 910 in the elastic layer 920. In some embodiments, at least a portion of the shell 910 is treated with a release agent so that the elastic layer 920 will not adhere to the shell 910. In this manner, radial forces required to cause the EGD 101 to expand from a first diameter to a second diameter will be minimized as displacement of the elastic layer 920 will be more uniformly distributed around the periphery of the shell 910.

FIG. 11 shows an embodiment similar to FIG. 9 with four shell segments 1110 and an elastic layer 1120.

FIG. 12 shows another embodiment, where the backing layer is formed from a plethora of shell segments 1210 with inner surface 1212 and an elastic layer 1220. The inner surface 1212 of the shell segment 1210 can generally be curved or flat depending on the embodiment.

FIG. 13 shows an embodiment similar to FIG. 12 where the shell segments 1310 are rod-like structures of a generally circular cross section.

FIG. 14 shows an embodiment, comprising a backing layer formed by at least two shell segments 1410 where the inner surface 1412 of one shell segment 1410 overlaps the outer surface 1414 of an adjacent shell segment 1410 and a continuous elastic layer 1420. The inner surface 1412 can generally be curved or flat depending on the embodiment. Upon insertion of a stenting device 151 into a lumen 1430 with an initial diameter D1-14, radial forces can be generated against the walls of the lumen 1430 causing the shell segments 1410 to slide and/or bend relative to each other and separate until the lumen 1430 expands to a final diameter D2-14 whereby the continuous layer 1420 generates a compressive radial force on the backing layer. Upon removal of the stenting device 151 from the lumen 1430, the compressive force generated by the continuous layer 1420 causes the shell segments 1410 to slide and/or bend relative to each other and approach each other until the lumen 1430 contracts to approximately its initial diameter D1-14. Those skilled in the art will recognize the embodiment of FIG. 14 can be realized with two or more shell segments 1410 of equal or variable arc lengths.

FIG. 15 shows an embodiment similar to FIG. 14 comprising a backing layer of shell segments 1510 where a portion of the inner surface 1512 of each shell segment 1510 overlaps a portion of the outer surface 1514 of an adjacent shell segment 1510 and a continuous elastic layer 1520. The inner surface 1512 and outer surface 1514 can generally be curved or flat depending on the embodiment. Upon insertion of a stenting device 151 into a lumen 1530 with an initial diameter D1-15, radial forces can be generated against the walls of the lumen 1530 causing the shell segments 1510 to slide and/or bend relative to each other and separate until the lumen 1530 expands to a final diameter D2-15 whereby the continuous layer 1520 generates a compressive radial force on the backing layer. Upon removal of the stenting device 151 from the lumen 1530, the compressive force generated by the continuous layer 1520 causes the shell segments 1510 to slide and/or bend relative to each other and approach each other until the lumen 1530 contracts to approximately its initial diameter D1-15. Those skilled in the art will recognize the embodiment of FIG. 15 can be realized with two or more shell segments 1510 of equal or variable arc lengths.

FIG. 16 shows an embodiment comprising a backing layer of shell segments 1610 with a generally circular cross-sectional area where the segments 1610 are disposed relative to each other using a tongue-and-groove arrangement and a continuous elastic layer 1620. Upon insertion of a stenting device 151 into a lumen 1630 with an initial diameter D1-16, radial forces can be generated against the walls of the lumen 1630 causing the tongue-and-groove segments to slide and/or bend relative to each other and separate until the lumen 1630 expands to a final diameter D2-16 whereby the continuous layer 1620 generates a compressive radial force on the backing layer. Upon removal of the stenting device 151 from the lumen 1630, the compressive force generated by the continuous layer 1620 causes the tongue-and-groove segments to slide and/or bend relative to each other and approach each other until the lumen 1630 contracts to approximately its initial diameter D1-16. Those skilled in the art will recognize the embodiment of FIG. 16 can be realized with two or more shell segments 1610 of equal or variable arc lengths. FIG. 17 shows an embodiment comprising a backing layer where, here, an inner shell 1710 and an outer shell 1715 are nested one within the other to form a lumen 1730 and a continuous elastic layer 1720. Upon insertion of a stenting device 151 into a lumen 1730 with an initial diameter D1-17, radial forces can be generated against the walls of the lumen 1730 causing the inner shell 1710 and the outer shell 1715 segments to slide and/or bend relative to each other and separate until the lumen 1730 expands to a final diameter D2-17 whereby the continuous layer 1720 generates a compressive radial force on the backing layer. Upon removal of the stenting device 151 from the lumen 1730, the compressive force generated by the continuous layer 1720 causes the inner shell 1710 and outer shell 1715 segments to slide and/or bend relative to each other and approach each other until the lumen 1730 contracts to approximately its initial diameter D1-17. Those skilled in the art will recognize the embodiment of FIG. 17 can be realized with two or more shell segments 1710 of equal or variable arc lengths.

FIG. 18 shows an embodiment comprising a backing layer where at least two shell segments here, a left shell 1810 and a right shell 1815, are nested to form a lumen 1830 and an elastic layer 1820. In FIG. 18, a portion of the inner surface of the left shell 1810 is over lapping a portion of the outer surface of the right shell 1815 and a portion of the inner surface of the right shell 1815 is over lapping a portion of the outer surface of the left shell 1810. Upon insertion of a stenting device 151 into a lumen 1830 with an initial diameter D1-18, radial forces can be generated against the walls of the lumen 1830 causing the shell segments 1810 and 1815 to slide and/or bend relative to each other and separate until the lumen 1830 expands to a final diameter D2-18 whereby the continuous layer 1820 generates a compressive radial force on the backing layer. Upon removal of the stenting device 151 from the lumen 1830, the compressive force generated by the continuous layer 1820 causes the shell segments 1810 and 1815 to slide and/or bend relative to each other and approach each other until the lumen 1830 contracts to approximately its initial diameter D1-18. Those skilled in the art will recognize the embodiment of FIG. 18 can be realized with two or more shell segments of equal or variable arc lengths.

FIG. 19 shows an embodiment comprising a backing layer of shell segments 1910 where an elongated portion of the inner surface 1912 of each shell segment 1910 overlaps an elongated portion of the outer surface 1914 of an adjacent shell segment 1910 and a continuous elastic layer 1920. The inner surface 1912 and outer surface 1914 can generally be curved or flat depending on the embodiment. Upon insertion of a stenting device 151 into a lumen 1930 with an initial diameter D1-19, radial forces can be generated against the walls of the lumen 1930 causing the shell segments 1910 to slide and/or bend relative to each other and separate until the lumen 1930 expands to a final diameter D2-19 whereby the continuous layer 1920 generates a compressive radial force on the backing layer. Upon removal of the stenting device 151 from the lumen 1930, the compressive forces generated by the continuous layer 1920 causes the shell segments 1910 to slide and/or bend relative to each other and approach each other until the lumen 1930 contracts to approximately its initial diameter D1-19. Those skilled in the art will recognize the embodiment of FIG. 19 can be realized with two or more shell segments 1910 of equal or variable arc lengths.

In another embodiment, the expanding sheath introducer 100 comprises a backing layer where a strip of backing material has been coiled into a shape similar to barber pole and an elastic layer. Depending upon the amount of overlap between segments and the pitch of the coil, radial resistance can be controlled to allow for easy insertion of the cannula tube 152 and subsequent expansion of the EGD 101. Note this embodiment also allows for uncoiling of the tube under tangential opposing forces directed along the coils of the tube applied at either end of the tube resulting in an expansion of tube diameter. The tube, segment overlap and coil pitch can all be controlled by manufacturing the tube with a thin strip of suitable backing material coiled around a mandrel and heated until the backing material assumes the form of the mandrel. The outer elastic layer is preferably loose around the coil tube (i.e., there is no adhesion between the outer elastic layer and coil tube) to minimize radial resistance.

In some embodiments, improved lubricity of all surfaces of the EGD 102 and stenting device 152 can be achieved by surface treatments including, but not limited to, coating or doping of the surfaces. In some embodiments, lubricity agents such as hydrophilic coatings, polytetrafluoroethylene (PTFE or Teflon), polysiloxanes with hydrophilic pendant groups, or parylene can be used as surface treatments.

In other embodiments, a coating, for example a lubricious, a hydrophilic, a protective, or other type of coating may be applied over portions or the entire device. Hydrophobic coatings such as fluoropolymers provide a dry lubricity which improves device exchanges. Lubricious coatings improve steerability and improve lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. In some embodiments, the sheath or coating may be applied over the basket region. This may provide extra surface area to contain clots that might be captured therein.

In some cases, surface treatments may change the local properties of the surfaces treated. In some embodiments where a relatively elastic element has been made relatively inelastic due to a surface treatment, relative elasticity can be restored by cutting a groove into or through the treated surface (e.g., scoring) to allow for expansion or contraction of the underlying untreated material. FIG. 20 shows an example where the elastic layer (or outside surfaces) of the EGD 102 are surface treated with materials to reduce surface friction, as in one example, polytetrafluoroethylene (PTFE). Since PTFE treatments can result in relatively elastic surfaces becoming relatively inelastic and thereby impeding EGD 102 expansion capabilities, the unexpanded, PTFE-treated elastic layer of the EGD 102 can be scored once or multiple times as shown in FIG. 20A. A scored line on a surface treated object can take numerous shapes including, but not limited to, longitudinal lines or spiral lines about the long axis of the EGD 102.

Referring again to FIG. 20A, an embodiment of the unexpanded, surface treated elastic layer of the EGD 102 with lumen 105 of initial diameter D1 is scored through the surface treatment in three locations (labeled A, B and C) respectively. FIG. 20B shows the expanded, surface treated elastic layer of the EGD 102 with lumen 105 of a second diameter D2 where expansion of the lumen 105 has occurred due to stretching of the elastic layer under radial forces applied to the lumen 105 at the scored locations (A, B and C).

In some examples, suitable polymeric materials may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example a polyetherester elastomer such as ARNITELB™ available from DSM Engineering Plastics), polyester (for example a polyester elastomer such as HYTRELB™ available from DuPont), polyamide (for example, DURETHANB™ available from Bayer or CRISTAMIDB™ available from Elf Atochem), elastomeric polyamides, block polyamideiethers, polyether block amide (PEBA, for example available under the trade name PEBAXB), silicones, polyethylene (PE), Marlex™ high density polyethylene, Marlex™ low-density polyethylene, linear low density polyethylene (for example REXELLB™), poly-ethylene terephthalate (PET), polyetheretherketone (PEEK), polyimide (PI), polyetherimide (PEI), polyphenylene sulfide (PPS), polyphenylene oxide (PPO), polysulfone, nylon, perfluoro(propyl vinyl ether) (PFA), other suitable materials, or mixtures, combinations, copolymers thereof, polymerimetal composites, and the like. In some embodiments, portions of or all of the device can be blended with a liquid crystal polymer (LCP). For example, the mixture can contain up to about 5% LCP.

The expanding sheath introducer 100, or portions thereof, may be formed, for example, by coating, electrophoresis, by extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

The expanding sheath introducer 100, or portions thereof, may also be coated, plated, wrapped or surrounded by, doped with, or otherwise include a radiopaque material. Radiopaque materials are understood to be materials capable of producing a relatively bright image on a fluoroscopy screen or another imaging technique during a medical procedure. This relatively bright image aids the user of the device in determining its location. Some examples of radiopaque materials can include, but are not limited to, gold, platinum, palladium, tantalum, tungsten alloy, plastic material loaded with radiopaque filler, and the like.

### Methods of Use

The expanding sheath introducer 100 uses the aforementioned Seldinger method to place a guidewire in a vesicle in preparation for insertion of a sheath introducer. According to one embodiment, a practitioner prepares a sheath 100 by inserting a distal end of a standard dilator (e.g., an elongate flexible cylinder with a bore extending therethrough) as noted in U.S. Pat. No. 6,183,443 into the proximal end portion 107 of the EGD 101 and advances the dilator fully into the EGD 101 until a distal tip portion of the dilator extends distally beyond a distal portion 109 of the EGD 101 and a proximal portion of the dilator remains outside the proximal end portion 107 of the EGD 101. The distal tip portion of the dilator has a tapered outer diameter that gradually increases proximally to a diameter that is approximately equal to the distal end portion 109 of the EGD 101. The proximal portion of the dilator is releasably secured to the proximal end portion 107 of the EGD 101 so that the dilator and EGD 101 comprise an assembled expanding sheath introducer 100 for insertion into the vessel.

FIG. 21 shows an embodiment of the EGD 101 with a distal end portion 109 that presents a square leading edge 2110 with the potential to catch on tissue as the expanding sheath introducer 100 is inserted into the vesicle. An introducer component 2120 with a teardrop-shaped tapered segment 2130 where the largest diameter of segment 2130 is approximately equal to the outer diameter of the EGD 101 can reduce the risk of catching. The taper of the segment 2130 presents a gradually increasing diametral cross-section to the vesicle and allows the practitioner to gently part and move tissue out of the way avoiding potential damage to the tissue that might be associated with the square leading edge 2110. Further, a tapered rear portion 2140 of the tapered segment 2130 allows a medical practitioner to more easily withdraw the introducer component 2120 over the square edge 2110 by presenting a gradually increasing diametral cross-section to gently part the distal end portion 109 to allow the segment 2130 to pass through the lumen 105 of the EGD 101.

FIG. 22 shows an embodiment of the EGD 101 with a distal end portion 109 that presents a tapered leading edge 2210. The tapered edge 2210 greatly reduces the potential that tissue may catch on the EGD 101 as the expanding sheath introducer 100 is inserted into the vesicle since the tapered edge 2210 presents a gradually increasing diametral cross-section to the vesicle that gently parts and moves tissue away from the advancing introducer 100. The tapered edge 2210 can be matched to commercially-available standard dilator components to present a smooth transition from the dilator to the EGD 101.

### Dilator-Less Embodiments

Some embodiments of the expanding sheath introducer 100 do not require a standard dilator to insert the device into a vesicle. FIG. 23 shows an example of an expanding sheath 100 comprising an EGD 101 and a tapered tip 2310. In one embodiment, the tapered tip 2310 can be formed by cutting a shape that is approximately rectangular on a proximal end and approximately triangular on a distal end from a thin sheet of material in the approximate dimensions of the EGD 101. The triangular section is then bent at an angle and a set introduced such that when the rectangular section is rolled into a tube, the point of the triangular section will be located approximately on the center axis of the rolled tube. Further, the edges of the rolled tube can be overlapped as shown in FIG. 7.

In another embodiment, the tapered tip 2310 can be formed from at least two segments attached to the distal end portion 109 of the EGD 101. FIG. 24 shows an embodiment of a tip shell 2410 with a tapered distal end 2420, tapered distal lumen 2430 and an untapered proximal end 2440. The tip shell 2410 can be made from an elastic material, a rigid material or a combination of both materials to minimize vessel damage on expansion of the EGD 101.

Referring again to FIG. 23, the tapered tip 2310 can be formed from two tip shells 2410 placed back-to-back to create an interface between the two shells 2410 and attached at the untapered proximal end 2440 to the distal end portion 109 of the EGD 101. In operation, the expanding introducer sheath 100 with tip shells 2410 is threaded onto a guidewire inserted into the vesicle by the aforementioned Seldinger method and advanced into the tissue. The tapered tip 2310 presents a gradually increasing diametral cross-section to the vesicle and allows the practitioner to gently part and move tissue out of the way allowing insertion of the sheath 100 into the vesicle. After withdrawing the guidewire, a stenting device 152 is introduced into the proximal end portion 107 of the EGD 101 and advanced through a lumen 105 to a distal end portion 109 and the tapered tip 2310 attached thereto. As the stenting device 152 is further advanced into the tapered tip 2310, the outside diameter of the stenting device 152 contacts the sides of the tapered distal lumen 2430 which creates a radial force that operates to separate the tapered tip 2310 at the interface formed by the two tip shells 2410 and allow a distal end portion 157 of the stenting device 152 to pass. The stenting device 152 is further advanced until the distal end portion 157 extends distally beyond the distal end portion 109 of the EGD 101. Those skilled in the art will recognize the embodiment of FIG. 23 can be realized with two or more tip shells 2410 of equal or variable arc lengths.

Stenting Device embodiments

In many percutaneous procedures, the introducer guidewire or other guidance device has a ‘free’ end (e.g., there is no instrument or impediment between the proximal end of the guidance device and its entrance into the vesicle) protruding from the vesicle over which a stenting device 152 or other instruments can be inserted. In other procedures, there are instruments or impediments between the proximal end of the guidance device and its entrance into the vesicle that will prevent introduction of a stenting device or other instrument into the sheath 100. Further, there can be situations when it is desirable to replace a first stenting device 152 with a second stenting device 152 of a different diameter, but the path for withdrawing the first device 152 and inserting the second device 152 over a guidance device is prevented by an instrument or otherwise blocked, for example, in transaortic valve implantation (or TAVI) procedures.

Further, devices that expand occupy a greater cross-sectional area of the vesicles into which they are inserted and can block blood flow in those vesicles creating a potential for thrombosis or other complications. It can be desirable to expand a sheath from a first diameter to a second larger diameter for insertion of diagnostic or therapeutic instruments and then retract the sheath from the second diameter to the first diameter to improve blood circulatory flow during a percutaneous procedure. Several different designs can allow for stenting device 152 replacements.

FIG. 25 shows an embodiment of a stenting device 2510 with a generally circular cross-section has a removed portion 2520 to allow the passage of a catheter into the lumen 158 of a stenting device 152. When combined with the EGD 101, the TAVI-like catheter and a hub 159, the device operates as follows (see Procedure A):

### Procedure A:

1. Insert an unexpended EGD 101 of a first diameter D1 into a vesicle of interest.

2. Insert a stenting device 152 to expand the EGD 101 from the first diameter D1 to a second diameter D2.

3. Insert a diagnostic or therapeutic catheter-like assembly through the stenting device 152 and into the vesicle.

4. Pull the stenting device 152 out of the EGD 101 and remove the stenting device 152 from around the diagnostic or therapeutic catheter-like assembly through a removed portion 2520 whereby the EGD 101 returns to the first diameter allowing for improved blood flow in the vesicle.

In order to generate radial forces required to expand the EGD 101 the stenting device 152 must be designed with sufficient care to prevent collapse of the removed portion 2520. The stenting device 152 can be constructed from a variety of materials including, but not limited to, stainless steel or other semi-rigid plastics. Further a hemostatic valve designed as a wiper to seal the lumen 158 of the removed portion 2520 of the stenting device 152 during insertion and removal will likely be required for the design of FIG. 25.

FIG. 26 shows an embodiment of the stenting device 152 with a generally circular cross section and a removable segment with the addition of a removable segment 2640. In this design, procedure A (see infra) is the same except a step 5 replaces step 4, specifically:

5. Pull the stenting device 152 with segment 2640 out of the EGD 101 and slide the segment 2640 out of the guide means 2650 to reveal the removed portion 2620.

Note the guide means 2650 can be any feature that will assure a linear path including, but not limited to slide rails internal to the wall thickness of the stenting device 152 or slide rail features external to the wall thickness of 152. FIG. 27 shows a shell-in-shell design as an example of an external feature.

This design has the advantage that standard hemostatic valves can be used in the outer sheath since removal of the stenting device 152 presents a closed section and thus, an ordinary sealing surface to the hemostatic valve.

FIG. 28 shows an embodiment of the stenting device 152 with a generally circular cross section and at least one weakened or breakaway seam 2850 to allow the cannula tube to be “broken off” the catheter by hand or tools commonly found in the operating theater.

FIG. 29 shows an embodiment of the stenting device 152 with a generally circular cross section and at least one gap 2950 and a live hinge 2960. By pulling open the sides of the gap 2950, the stenting device 152 can be fitted around or removed from the diagnostic or therapeutic catheter-like assembly. Upon insertion into the EGD 101, the stenting device 152 will be in compression thereby closing the gap creating a sealing surface for the EGD hub 111 to allow the device 152 to be removed from the EGD 101 with minimal blood loss.

FIG. 30 shows an embodiment of the stenting device 152 with a gap cover 3050 attached to segment 3060 and overlapping segment 3065 to cover the gap 3070. By pulling open the sides of the gap 3070, the live hinge 3080 will bend so the stenting device 152 can be fitted around or removed from the diagnostic or therapeutic catheter-like assembly. After release of the gap opening force, the live hinge 3080 will cause the stenting device 152 to regain its generally circular cross-section. Upon insertion into the EGD 101, the stenting device 152 will be in compression thereby closing the gap 3070 and pressing the gap cover 3050 tightly against segments 3060 and 3065 to create a sealing surface for the EGD hub 111 thus allowing the device 152 to be inserted or removed from the EGD 101 with minimal blood loss

In another embodiment, gap cover 3050 can be a tape-like structure that covers the gap 3070 and adheres to both segments 3060 and 3065. In one method of use, the embodiment shown in FIG. 30 is the same as procedure A (infra) except step 4 is replaced by step 6, specifically:

6. Pull the stenting device 152 out of the EGD 101 and withdraw it until it hangs loose on the diagnostic or therapeutic catheter-like assembly. Remove the tape-like gap cover 3050 to expose gap 3070 and bend segments 3060 and 3065 apart to remove the stenting device 152 from the catheter-like assembly.

### Self-Stenting EGD Embodiments

Other embodiments of an expanding sheath introducer 100 can be realized with a self-stenting design where the requirement of a stenting device 152 is not required to maintain the lumen 105 of the EGD 101 at a desired diameter. FIG. 31 shows a thin sheet of material 3105 into which a series of V-shaped perforations 3110 have been created. FIG. 32 shows the structure of one of the V-shaped perforations 3110 comprising a tooth 3120 and a corresponding hole 3130. FIG. 33 shows a section view of a V-shaped perforation 3110 where the tooth 3120 has been formed in a curved fashion and set such that a tooth bent into the plane of the thin material 3105 will naturally return to its curved position after release of the bending force. The tooth 3120 generally creates a tooth angle 3140 with respect to the plane of thin material 3105.

A ratchet-like embodiment of a self-stenting EGD 101 comprising a backing layer and an elastic layer can be formed by rolling the thin material 3105 into a tube of a first diameter D1 where the long edges overlap a sufficient portion of the material 3105 in a fashion similar to the cross section shown in FIG. 7. The tube is rolled such that the tooth angle 3140 is directed towards the middle of the tube. The elastic layer generates a compressive force on the material 3105 to help prevent the tube from unfurling and positions the teeth 3120 so they are generally compressed against the tube of thin material 3105. When a mandrel or other device with a diameter larger than the first diameter D1 is inserted into the lumen of the tube, the tube will expand to a second diameter D2 under the radial force created by the mandrel moving through the tube lumen as the teeth 3120 slide across the holes 3130 in the overrun direction as shown in FIG. 32. As the mandrel is withdrawn, the elastic layer applies a compressive force to the outside of the tube causing the tube to retract in the ratchet direction as shown in FIG. 32, however, as the teeth 3120 pass over the holes 3130, the teeth 3120 assume their naturally curved form thereby extend into the holes. As the tube continues to move in the ratchet direction under the compressive forces of the elastic layer, the teeth 3120 lock into the holes 3130 in a ratchet-like fashion and prevent the tube from advancing any farther in the ratchet direction.

In another embodiment, the tube material 3110 is rolled such that the tooth angle 3140 is directed away from the middle of the tube.

### Infection Protection/Inhibiting Embodiments

FIG. 34 shows an embodiment where a repeating surface texturing (two instances of the repeated pattern shown) that can be applied to any surface of the device to minimize the growth of infection causing organisms. This surface treatment is similar to the commercialized Sharklet technology (see http://www.sharklet.com/)

FIG. 35 shows an embodiment where the EGD 102 design is used as a waveguide for radiation (such as UV light) to kill infection causing organisms. A radiation source 3510 is applied to the waveguide end 3520 and the radiation irradiates the EGD 101 surface along the waveguide sterilizing it. The EGD 101 of FIG. 35 is made by the process of 1) obtaining a tube-like piece of material appropriate to transmit radiation along its length (See FIG. 36A); 2) flattening a portion of the tube-like piece and trimming appropriately (See FIG. 36B); and 3) rolling the flattened portion into a tube that may overlap so that the waveguide head and rolled tube are as shown in the side and end views (See FIG. 36C). FIG. 36D shows typical side (left) and end (right) views of the radiation waveguide tube of FIG. 35.

### Blood Loss Prevention Embodiments

While most vessel walls have an inherent elasticity, the vessels of infirm or elderly patients may not recover as quickly from expansion of a vessel penetration. In these cases, as the previously expanded sheath is collapsed, the vessel seal against the sheath will not be maintained and loss of blood through seepage around the penetration will occur. To prevent this loss of blood, a tools and/or techniques designed to gather the edges of the vessel and pull them into close proximity to the sheath such as the tool in U.S. Pat. No. 4,605,002 and/or manual suture methods can be applied.

By way of non-limiting examples, the present technology may include a device to allow access into a body having at least:

A first cannula with an outer surface, an inner lumen surface, an inner lumen diameter and proximal and distal end portions; and

An expandable covering over the outer surface, the expandable covering having an outer surface and an inner surface;

The inner surface of the expandable covering remaining in contact with the outer surface of the first cannula.

The device may have a tubular medical device which resides within the first cannula, the tubular medical device having a length and having diameter thicknesses along its length, the expandable covering applying a compression force against the first cannula, and the inner surface of the first cannula being held in contact against the outer surface of the tubular medical device. The expandable covering may be at least an elastomeric polymer tube and the first cannula comprises a polymeric cannula having less elasticity than the expandable covering. The first cannula may be at least a segmented polymeric tube, the segments allowing the first cannula to remain in contact with the expandable covering as the expandable covering expands by the first cannula separating between sections of itself to change the inner lumen diameter. The first cannula may be at least a polymeric tube, and the inner lumen surface is smooth, allowing for passage of tubular elements through the length of the first cannula. The first cannula should have a higher Young's Modulus and greater stiffness along its length than does the expandable covering.

A method of using the device described herein may include inserting a tubular medical element into a vessicle by at least

a) inserting the tubular medical element, having an outer surface thereon, into a delivery device to allow access through the delivery device and through a vesicle, the delivery device comprising:
a first cannula with an outer surface, an inner lumen surface, an inner lumen diameter and proximal and distal end portions; and an expandable covering over the outer surface, the expandable covering having an outer surface and an inner surface;

b) advancing the tubular medical device through the first cannula, exerting a force against the inner lumen surface from the outer surface of the tubular medical element; and

c) expanding the expandable cover with force transmitted from the tubular medical device through the first cannula.

In the method, after steps a), b) and c) have been performed with a first tubular medical device having a first average diameter, withdrawing the first tubular media device from the first cannula, and performing steps a), b) and c) with a second tubular medical device that has a second average diameter different from the first average diameter of the first tubular medical device.

The device may further have at least a hub (valve) attached to said proximal end portion of said first cannula. The device may also have at least a second cannula with an outer surface disposed such that when said second cannula is inserted into said inner lumen of said first cannula said outer surface of said second cannula slides against said inner lumen surface of said first cannula thereby preserving the initial or expanding said inner lumen diameter of said first cannula.

According to one embodiment, the tube for a sheath introducer according to the disclosure may be manufactured by a procedure including at least:
a) First forming the internal layer by molding, extrusion, sheet formation, conversion, bending prepared sheets, rolling and fusing a bent prepared sheet, or otherwise using known manufacturing techniques to form the internal, less flexible, more longitudinally stiff element of the device. The outer flexible layer(s) of the device may then be coated on, shrunk on, slid over, wrapped over, fabricated over, woven over, knitted over, or otherwise engaged on the outer surface of the inner element.
b) The device may also be constructed in a reverse order from a), above. The outer layer (the more flexible layer) may be first formed and the inner, more longitudinally rigid layer may be formed or inserted into the outer more flexible layer. This may be done by an internal coating method 9such as dip coating with contact-retained hardenable material on only the inner surface of the outside more flexible element, spin coating on the inside surface of the outer element, sliding the internal element into the outer element and reshaping or resizing the inner element (e.g., by unfolding it, stretching it and rehardening it, or other know methods.
c) The device may also be formed by coextruding the two layers through a multi-slot die.
d) The device may also be formed by coextruding the two layers through a multi-slot cylindrical die to form the two layers contemporaneously.
e) The device may also be formed by first forming a sheet of the two layers, by coating or a release surface, forming one layer then extruding the other layer over it, coextruding a sheet of the two materials, and then converting (cutting) the two layer sheet and rolling the converted elements into cylindrical devise and securing the edges of the converted sheet where needed.
   Other known types of methods of manufacture may be used. It is desirable, but not essential, that the two layers of the device be secured to each other. This may be done by tension, fusion, adhesives or the like. If an adhesive is used, this would essentially describe a three-layer device, with an adhesive layer between the inner longitudinally more rigid layer and the outer more flexible layer.

It will be understood that the embodiments which have been described are illustrative of some of the applications of the principles of the disclosure. Numerous modifications may be made by those skilled in the art without departing from the true spirit and scope of the disclosure. Various features which are described herein can be used in any combination and are not limited to procure combinations that are specifically outlined herein.

What is claimed:

1. A device to allow access into a body comprising:
A first cannula with an outer surface, an inner lumen surface, an inner lumen diameter and proximal and distal end portions, and;
An expandable membrane covering at least a portion of said outer surface of said first cannula.

2. A device to allow access into a body comprising:
A first cannula with an outer surface, an inner lumen surface, an inner lumen diameter and proximal and distal end portions; and
An expandable covering over the outer surface, the expandable covering having an outer surface and an inner surface;
The inner surface of the expandable covering remaining in contact with the outer surface of the first cannula.

3. The device of claim 2 wherein a tubular medical device resides within the first cannula, the tubular medical device having a length and having diameter thicknesses along its length, the expandable covering applying a compression force against the first cannula, and the inner surface of the first cannula being held in contact against the outer surface of the tubular medical device.

4. The device of claim 3 wherein the expandable covering comprises an elastomeric polymer tube and the first cannula comprises a polymeric cannula having less elasticity than the expandable covering.

5. The device of claim 4 wherein the first cannula comprises a segmented polymeric tube, the segments allowing the first cannula to remain in contact with the expandable covering as the expandable covering expands by the first cannula separating between sections of itself to change the inner lumen diameter.

6. The device of claim 4 wherein the first cannula comprises a polymeric tube, and the inner lumen surface is smooth, allowing for passage of tubular elements through the length of the first cannula.

7. The device of claim 6 wherein the first cannula has a higher Young's Modulus and greater stiffness along its length than does the expandable covering.

8. A method of inserting a tubular medical element into a vessicle comprising
a) inserting the tubular medical element, having an outer surface thereon, into a delivery device to allow access through the delivery device and through a vesicle, the delivery device comprising:
a first cannula with an outer surface, an inner lumen surface, an inner lumen diameter and proximal and distal end portions; and an expandable covering over the outer surface, the expandable covering having an outer surface and an inner surface;
b) advancing the tubular medical device through the first cannula, exerting a force against the inner lumen surface from the outer surface of the tubular medical element; and
c) expanding the expandable cover with force transmitted from the tubular medical device through the first cannula.

9. The method of claim 8 wherein after steps a), b) and c) have been performed with a first tubular medical device having a first average diameter, withdrawing the first tubular media device from the first cannula, and performing steps a), b) and c) with a second tubular medical device that has a second average diameter different from the first average diameter of the first tubular medical device.

10. The device according to claim 2 further comprising a hub (valve) attached to said proximal end portion of said first cannula.

11. The device according to claim 2 further comprising a second cannula with an outer surface disposed such that when said second cannula is inserted into said inner lumen of said first cannula said outer surface of said second cannula slides against said inner lumen surface of said first cannula thereby preserving the initial or expanding said inner lumen diameter of said first cannula.
